(19) United States
(12) Patent Application Publication
(10) Pub. No.: US 2006/0188450 A1
(43) Pub. Date: Aug. 24, 2006

(54) SYSTEM AND METHOD FOR SUPPRESSING A COUGH

(76) Inventor: Tim Clarot, Phoenix, AZ (US)

Correspondence Address: SNELL & WILMER;ONE ARIZONA CENTER, 400 EAST VAN BUREN, PHOENIX, AZ, 85004-2202, US

(21) Appl. No.: 11/276,352

(22) Filed: Feb. 24, 2006

Related U.S. Application Data
(60) Provisional application No. 60/593,922, filed on Feb. 24, 2005.

(51) Int. Cl. A61K 31/137; A61L 9/04

(52) U.S. Cl. 424/45; 514/649

(57) ABSTRACT

A system and method for suppressing coughs, congestion and related symptoms is provided. The composition sprayed into the oral cavity may contain a cough suppressant and other ingredients such as decongestants, flavorings, sweeteners, stablizers, and/or preservatives. The composition is sprayed into the oral cavity utilizing a spray pattern having a spray pattern ratio from about 1.0 to about 1.19. The method of application comprises multiple applications of the composition every four to six hours according to condition severity and a user's age characteristics.

### CROSS-REFERENCE TO RELATED APPLICATIONS

This application is a non-provisional of U.S. Provisional Application Ser. No. 60/593,922 filed Feb. 24, 2005 and is incorporated herein by reference.

### FIELD OF INVENTION

The present invention relates generally to a system for suppressing a cough, and more particularly, to a system for effectively delivering a uniform dosage of a formulated composition for suppressing coughs.

### BACKGROUND OF THE INVENTION

Dextromethorphan (DXM) is a commonly used medicant for suppressing coughs. DXM is known to suppress the area of the brain that causes coughing. DXM is found in many commercial products, such as, for example, Robitussin Cough Calmers, Robitussin Maximum Strength, Scot-Tussin DM Cough Chasers, St. Joseph Cough Suppressant, Vicks 44 Cough Medicine and Vicks Formula 44, to name a few.

Oral administration of pharmaceuticals is common due to the increased absorption characteristics of the oral cavity. As such, a wide variety of oral delivery mechanisms exist for pharmaceuticals. For example, pills or tablets may be administered orally, but these mechanisms may prove difficult for children and others who have difficulty swallowing. Liquid formulations are also available; many times, however, these formulations are disagreeable to the palate and may require preparation and clean up.

Thus, there is a need for a palatable, portable, and easily renderable oral dosage system for effectively delivering a uniform dosage of a formulated composition for suppressing coughs.

### SUMMARY OF THE INVENTION

In accordance with various exemplary embodiments of the present invention, as further described in greater detail below, an oral spray composition in accordance with one aspect of the present invention comprises a low viscosity cough suppressant formulation configured to be administered to the oral cavity via a fine spray mist in a dosage of about four squirts, each containing approximately 250 microliters. Preferably, in some treatments, the preferred dosage is advantageously administered approximately every four hours.

In accordance with various other aspects of the invention, a system for delivering the composition to the oral cavity is provided. More specifically, a spray applicator is provided for administering spray into the oral cavity.

These and other advantages of the various compositions, methods and systems according to various aspects of the present invention will be apparent to those skilled in the art upon reading and understanding the detailed description below.

### BRIEF DESCRIPTION OF THE DRAWINGS

Additional aspects of the present invention should become evident upon reviewing the non-limiting embodiments described in the specification taken in conjunction with the accompanying figures, wherein like numerals designate like elements, and:

FIG. 1 is a perspective view of an exemplary embodiment of a spray dispenser of the present invention.

### DETAILED DESCRIPTION

The following descriptions are of exemplary embodiments of the invention only, and are not intended to limit the scope, applicability or configuration of the invention in any way. Rather, the following description is intended to provide a convenient illustration for implementing various embodiments of the invention. As will become apparent, various changes may be made in the arrangement of elements described herein without departing from the scope of the invention. For example, though not specifically described, variations in the shape and orientation of the spray system should be understood to fall within the scope of the present invention. Further, variations in the contents of the spray system should also be understood to fall within the scope of the present invention as long as an appropriate spray pattern is maintained as defined in the claims.

A preferred oral spray composition of the present invention preferably reduces the presence and/or duration of symptoms associated with the common cold including, but not limited to, coughing and congestion. While other now known or hereafter developed formulations may also advantageously be used in accordance with various teachings of the present invention, preferably the oral spray composition of the present invention comprises a medicant which suppresses a cough and/or congestion for a period of time. The oral spray may be used from any time when an individual first notices any signs of a cold up until when the symptoms have cleared.

In accordance with various embodiments of the present invention, the medicant contains Dextromethorphan (DXM) HBR in an amount from about 0.7 to about 1.3 weight percent of the composition. In an exemplary embodiment, the DXM HBR composition comprises about 0.9 to about 1.1 weight percent of the oral spray composition.

In other embodiments of the present invention, the medicant contains a decongestant. The decongestant may include any suitable decongestant such as, for example, phenylephrine or the like. In an exemplary embodiment, the decongestant comprises phenylephrine present at an amount of about 0.7 to about 1.3 weight percent, preferably 0.9 to about 1.1 weight percent of the oral spray composition.

The medicant may contain a mixture of ingredients suitable for the treating multiple symptoms. In accordance with various embodiments, the medicant may contain mixtures of cough suppressants, decongestants and the like. In accordance with various embodiments, the medicant contains a mixture of dextromethorphan and phenylephrine.

In accordance with one embodiment of the present invention, the oral spray composition also includes a pharmaceutically acceptable carrier. The pharmaceutically acceptable carrier may include any suitable fluid or liquid such as, for example, purified water, or the like. Preferably, the pharmaceutically acceptable carrier is present in an amount of about 75 to about 85 weight percent of the oral spray composition.

The oral spray composition may also include a stabilizer. In an exemplary embodiment, the stabilizer comprises glycerin present at an amount of about 0.5 to about 1.5 weight percent, preferably about 0.9 to about 1.1 weight percent of the oral spray composition, or any other substance which aids in keeping the DXM in an ionic form.

Sweetener and flavor enhancers may also be included in the oral spray composition. Sweeteners may include, among other things, fructose, sucrose and/or the like. Non-artificial sweeteners including sucralose and saccharine may be used. Preferably, when present, such additives may be present in an amount of about 15 to about 25 weight percent of the oral spray composition, for example on the order of about 20 weight percent of the oral composition. A variety of flavorings may be used. Preferably, when used, such are employed in a stable form of an extract. One exemplary embodiment of the oral spray composition includes a flavor enhancer, such as cherry or honey, for example, in an amount of about 0.9 to about 1.2 weight percent of the oral spray composition, for example, on the order of about 1 weight percent of the oral composition.

The invention further contemplates encapsulation for flavor masking, and/or any other method known herein and or to be developed for making the formulation more palatable. While flavoring and/or sweeteners may be preferred, the present invention contemplates omission of a flavoring, encapsulation and/or other flavor masking techniques.

In accordance with another aspect of one embodiment of the present invention, a preservative may be added to the oral composition. Any suitable preservative may be used in accordance with the present invention such as, for example, benzalkonium chloride, benzyl alcohol, and disodium EDTA. However, in general any material that facilitates stability of the various ingredients may be employed as a preservative. In one aspect of this embodiment, the preservative preferably includes a 50% solution of benzalkonium chloride admixed into the oral composition at a concentration of about 0.01 to about 0.02 percent by weight, preferably about 0.015 percent by weight.

An alcohol may also be included in various embodiments of the present invention. The alcohol may be used to facilitate a calming effect for night-time dosage. Any suitable alcohol may be used in accordance with the present invention such as, for example, benzyl alcohol. In one embodiment, the alcohol includes a solution of benzyl alcohol admixed into the oral composition at a concentration of about 9.5 to about 11 percent by weight, preferably about 10 percent by weight.

The composition of the present invention is preferably delivered to the oral cavity through the mouth by an applicator. Those skilled in the art will appreciate that any suitable applicator may be used, but preferably, the applicator is suitably configured to deliver the formulation to the oral cavity in a fine mist. In accordance with a preferred embodiment, the applicator is one available from Pfeiffer of America, 12 Roszel Road, Suite C-104, Princeton, N.J. 08540, under model number 13965.

The composition may be delivered to an individual in any suitable dosage as described herein. In accordance with one embodiment of the present invention, the oral spray applicator is configured to administer a unit dosage of about 0.25 ml of composition to the individual each time a pump associated with the spray applicator is activated (0.25 ml/spray). Preferably, the composition is delivered by applying about three to four sprays in the mouth approximately every four to six hours, more preferably 4 sprays every 6 hours and optimally on the order of about 3 to 4 sprays every 6 hours until the cold symptoms have subsided.

### EXAMPLES

The Examples set forth below are illustrative of various aspects of certain exemplary embodiments of the present invention. The compositions, methods and various parameters reflected therein are intended only to exemplify various aspects and embodiments of the invention, and are not intended to limit the scope of the claimed invention.

### Example 1

An exemplary oral spray composition for delivering an DXM to the oral cavity to reduce the presence of a cough is prepared by admixing the following ingredients:

| Component | Function | Amount % w/w |
|---|---|---|
| Dextromethorphan (DXM) | Active Ingredient | ˜1.0% |
| Benzalkonium Chloride | Preservative (Quaternary | <1.0% |
| | Ammonium Compound) | |
| Benzyl Alcohol | Preservative | <1.0% |
| Glycerin | Stabilizer | <1.0% |
| Sucralose | Sweetener | ˜20% |
| Water | Carrier | q.s. |

### Example 2

An exemplary oral spray composition for delivering an active DXM and phenylephrine compound to the oral cavity is prepared by admixing the following ingredients:

| Component | Function | Amount % w/w |
|---|---|---|
| Dextromethorphan (DXM) | Active Ingredient | ˜1.0% |
| Phenylephrine | Active Ingredient | ˜1.0% |
| Benzalkonium Chloride | Preservative (Quaternary | <1.0% |
| | Ammonium Compound) | |
| Benzyl Alcohol | Preservative | <1.0% |
| Glycerin | Stabilizer | <1.0% |
| Sucralose | Sweetener | ˜20% |
| Cherry Flavoring | Flavor Enhancer | ˜1.0% |
| Water | Carrier | q.s. |

### Example 3

An exemplary oral spray composition for delivering an active DXM and phenylephrine compound to the oral cavity of a child under the age of 12 years is prepared by admixing the following ingredients:

| Component | Function | Amount % w/w |
|---|---|---|
| Dextromethorphan (DXM) | Active Ingredient | ˜1.0% |
| Phenylephrine | Active Ingredient | <1.0% |
| Benzalkonium Chloride | Preservative (Quaternary | <1.0% |
| | Ammonium Compound) | |
| Benzyl Alcohol | Preservative | <1.0% |
| Glycerin | Stabilizer | <1.0% |
| Saccharine | Sweetener | ˜20% |
| Cherry Flavoring | Flavor Enhancer | ˜1.0% |
| Water | Carrier | q.s. |

An oral spray composition for reducing the severity and duration of symptoms of the common cold has been presented. The oral spray preferably is delivered by way of a fine spray mist through the use of a spray applicator. The formulation included in the oral spray preferably functions to suppress coughs and reduce coughing symptoms and optimally includes DXM. The formulation included may also reduce congestion in nasal and oral cavities.

The present invention contemplates various embodiments wherein the dosage for each embodiment various by age and/or cough severity. For example, in one embodiment, the average dosage contemplated for an adult (over 12 years) is 10 mg every four hours (approximately three sprays). The invention contemplates repeat dosage every four to six hours not to exceed 120 mg every 24 hours. In another embodiment, for children under the age of 12 years, the invention contemplates dosage that varies by age group. For example, the recommended dosage for children ages 6 to 12 years is about 5 to about 7.5 mg, not to exceed 60 mg in a twenty-four hour period. The recommended dosage for children ages 2 to 6 years is about 5 mg, not to exceed 30 mg in a twenty-four hour period.

In accordance with an exemplary embodiment of the present invention, a suitable system comprises a spray dispenser and a cough suppressant. The cough suppressant formulation in accordance with one of the various embodiments described in greater detail hereinabove is placed into a spray dispenser suitable for distributing the composition into an oral cavity. The spray dispenser may comprise any manually operated or automated means of producing a mist of liquid droplets, such as, for example, trigger-type, pump-type, non-aerosol self-pressurized, and aerosol-type spray means.

Preferably, when used as a cough suppressant, in accordance with any of the above-described formulations, the suppressant is dispensed such that the droplet size, dispersion angle, dispersion pattern, dispersion volume and/or dispersion velocity enable effective dosage of the cough suppressant with a minimum number of activations of the spray dispenser.

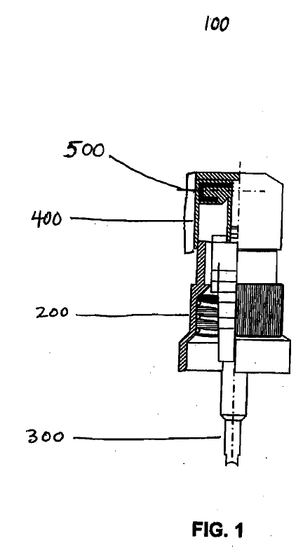

In accordance with an exemplary embodiment of the present invention, and with reference to FIG. 1, an oral spray pump 100 available from Pfeiffer of America, Princeton, N.J., is utilized because of the fine uniform spray characteristics, spray volume, and spray pattern size and shape. Spray pump 100 is placed inside a suitable bottle or container and such as, for example, is sealed by using the screw closure 200. In other embodiments, however, for example, a seal may be provided by any other suitable closures now known or hereafter devised, for example, use of other suitable enclosures which snap onto top of a bottle or container or other devices providing a secure fit. As spray pump 100 is placed into a container, a dip tube 300 is inserted into the container and through the composition contained therein. Upon depressing the spray actuator 400, the composition is drawn up by dip tube 300 and expelled through the spray nozzle 500. Spray nozzle 500 is configured such that a fine mist is produced with desirable spray characteristics, spray volume, and spray pattern and shape.

For example, in an exemplary embodiment of the present invention, upon depressing spray actuator 400, spray nozzle 500 delivers a spray pattern having a small diameter of about 34 mm to about 38 mm for example on the order of about 36 mm. Furthermore, spray nozzle 500 may deliver a spray pattern having a large diameter of about 34 mm to about 43 mm, for example on the order of about 39 mm. Although described as an exemplary embodiment, any spray nozzle and spray actuator that delivers a spray pattern in accordance with the present invention is suitable.

Accordingly, for example, spray nozzle 500 may have a spray pattern ratio of about 0.5 to about 1.30. As used herein, a spray pattern ratio refers to the ratio of the large spray diameter to the small diameter. Spray nozzle 500 may also be configured to have a spray angle of about 59 degrees to about 67 degrees. Although the spray angle is described with respect to spray nozzle 500, any spray dispenser delivering a spray angle in accordance with the present invention is suitable As used herein, the spray angle a is determined by the diameter of the spray pattern by the following equation:
α=2*arc tan (a/b)

a=½ diameter of the spray pattern

b=spray distance

Spray distance refers to the distance measured from the device to the spray surface, for example, the front of the oral cavity. For example, the distance is measured from, the side of spray actuator 400 where spray nozzle 500 expels composition to the spray surface. In a preferred embodiment of the present invention, the spray distance, b, is about 3 cm.

In another embodiment of the present invention, an oral spray pump may have an actuation velocity between 5 and 20 mm/s.

Any suitable bottle or container can be used with the sprayer dispenser. In an exemplary embodiment of the present invention, compositions formulated in accordance with the present invention and tinted in accordance with the above-described exemplary aspects, can be packaged in natural high-density polyethylene (HDPE), clear polyethylene terephthalate (PET) or polyvinylchloride (PVC) plastic bottles, allowing the tinted compositions to be visible through the packaging.

The present invention has been described above with reference to exemplary embodiments and examples. It should be appreciated that the particular embodiments shown and described herein are illustrative of the invention and its best mode and are not intended to limit in any way the scope of the invention as set forth in the claims. Those skilled in the art having read this disclosure will recognize changes and modifications may be made to the exemplary embodiments without departing from the scope of the present invention. For example, artisans will recognize that reference to oral, oralpharyngeal, and nasal membranes includes any interior surface of the oral and nasal cavities permitting delivery of an active substance, such as the DXM formulation, to the body, including the epithelial layer of the membranes or mucous of the epithelial layer of the membranes. Additionally, although certain components were described herein as being included in the oral composition in addition to the DXM formulation and the pharmaceutically acceptable carrier, it should be understood that carrier may be referred to as also including those certain components and that any suitable carrier may be achieved through any number of combination of additives now known or hereinafter devised. Accordingly, these and other changes or modifications are intended to be included to be within the scope of the present invention.

What is claimed is:

1. A system for suppressing coughs, congestion and related symptoms comprising:
a composition comprising
a medicant selected from the group of dextromethorphan present in an amount from about 0.7 to about 1.3 weight percent of the oral spray composition, decongestant, and mixtures thereof; and
an ingredient selected from the group of alcohol, stabilizer, preservative, sweetener, flavoring, and mixtures thereof; and
a spray dispenser configured to spray said composition into the oral cavity in at least one dosage amount said dispenser configured to deliver from about 0.1 to about 20 mg of medicant per said dosage amount.

2. The system of claim 1, wherein said dextromethorphan is present in an amount from about 0.9 to about 1.1 weight percent of the composition.

3. The system of claim 1, wherein said decongestant is phenylephrine present in an amount from about 0.7 to about 1.3 weight percent of the composition.

4. The system of claim 3, wherein said phenylephrine is present in an amount from about 0.9 to about 1.1 weight percent of the composition.

5. The system of claim 1, wherein said alcohol is present in an amount from about 9.5 to about 11 weight percent of the composition.

6. The system of claim 1, wherein said stabilizer is glycerin in an amount from about 0.9 to about 1.1 weight percent of the composition.

7. The system of claim 1, wherein said composition further comprises a preservative in an amount from about 0.01 to about 0.02 weight percent of the composition.

8. The system of claim 1, wherein spray dispenser has a spray pattern with a large diameter, A, and a small diameter, and wherein said dispenser has a spray distance, B, measured from the spray pump head of said dispenser to a spray surface, and wherein said dispenser has a spray angle, C, calculated as 2*arctan (A/B), wherein C is from about 59 to about 67 degrees.

9. The system of claim 8, wherein said spray pattern has a small diameter from about 34 to about 38 mm, and has a large diameter from abut 34 to about 43 mm.

10. The system of claim 9, wherein said spray pattern has a spray pattern ratio from about 1.0 to about 1.19.

11. The system of claim 8, wherein said dosage amount delivered is from about 0.15 to about 0.30 ml.

12. The system of claim 11, wherein said dosage amount delivered is from about 0.20 to about 0.25 ml

13. The system of claim 11, wherein said medicant per dosage amount delivered is from about 2.5 to about 15 mg.

14. The system of claim 13, wherein said medicant per dosage amount delivered is from about 5 to about 12 mg.
